(19) United States
(12) Patent Application Publication
(10) Pub. No.: US 2026/0311872 A1
(43) Pub. Date: Oct. 8, 2026

(54) SYSTEM FOR PROVIDING ARTIFICIAL INTELLIGENCE-BASED RETINAL DISEASE DIAGNOSIS ASSISTANCE SERVICE

(71) Applicant: Lux Mind Co., Ltd., Incheon (KR)

(72) Inventors: Duck Jin HWANG, Seoul (KR); Jee Woo Yoon, Seoul (KR); Seong Choi, Seoul (KR); Jun Seo Ko, Seoul (KR)

(73) Assignee: Lux Mind Co., Ltd., Incheon (KR)

(21) Appl. No.: 19/171,942

(22) Filed: Apr. 7, 2025

(51) Int. Cl. G16H 50/20; G06T 7/00; G06T 7/73; G06V 10/764; G06V 10/82; G16H 30/40

(52) U.S. Cl. CPC G16H 50/20; G06T 7/0012; G06T 7/73; G06V 10/764; G06V 10/82; G16H 30/40; G06T 2207/10101; G06T 2207/20084; G06T 2207/30041; G06T 2207/30096; G06V 2201/03

(57) ABSTRACT

Provided is a system for providing an artificial intelligence (AI)-based retinal disease diagnosis assistance service. The system includes a user terminal configured to transmit tomographic images of a retina captured by an optical coherence tomography (OCT) device, which uses a non-destructive examination method, and receive and output a retinal disease classification result and a diagnosis assistance service provision server that includes a preprocessing unit configured to receive and preprocess the tomographic images, a diagnosis assistance unit configured to input the preprocessed tomographic images into a prebuilt AI algorithm, a visualization unit configured to visualize a retinal disease classified by the prebuilt AI algorithm, and a sharing unit configured to provide the retinal disease classified by the prebuilt AI algorithm to the user terminal and transmit a visualization result.

### BACKGROUND

### 1. Field of the Invention

The present invention relates to a system for providing an artificial intelligence (AI)-based retinal disease diagnosis assistance service, and more particularly, to a system for preprocessing a brightness (B)-scan image captured using optical coherence tomography (OCT), detecting a retinal disease and classifying it into a retinal disease class, and then visualizing detection and classification results.

### 2. Discussion of Related Art

The retina, which is connected to the optic nerve, is an important sensory organ that is difficult to repair and inevitably leads to vision loss when damaged. The causes of retinal diseases have not been fully discovered, but they are thought to be mainly caused by diabetic retinopathy, hypertensive retinopathy, and the like. The number of patients diagnosed with retinal detachment, retinal vascular occlusion, and other retinal disorders continues to increase, with a high prevalence in people over 50, and the number of patients with retinal diseases is expected to continuously increase due to the elderly population and the rising number of patients with diabetes and hypertension. Metabolic syndrome, which predominantly occurs in middle-aged and older adults, is also associated with decreased retinal artery diameters. Among metabolic syndrome factors, abdominal obesity is particularly associated with decreased retinal artery diameters, arteriovenous niches, and the presence of retinal exudates, and is reported to have close association with retinal vascular changes. Eye diseases are classified as emergencies due to permanent vision impairment that may occur when treatment is delayed, and thus early diagnosis is very important.

Here, a method of diagnosing retinal diseases or detecting central serous chorioretinopathy (CSC) on the basis of deep learning has been researched and developed. In this regard, Korean Patent Registration No. 2023-2623722 (published on Jan. 11, 2024) and Korean Patent Application No. 2023-0168491 (published on Dec. 14, 2023) disclose a configuration for detecting retinal pigments in a retinal fundus image rather than an optical coherence tomography (OCT) image, and diagnosing a retinal disease on the basis of deep learning, and a configuration for preprocessing medical data to detect CSC using an artificial intelligence (AI) model and inputting the medical data into a pretrained neural network to detect and predict CSC, respectively.

However, neither the former nor latter configuration analyzes output results using OCT, but both simply interpret a retinal fundus image. OCT is a medical imaging technology for emitting near-infrared light onto the retina, measuring the difference in intensity between the emitted light and the reflected light and the time that it takes for the emitted light to bounce back, and obtaining high-resolution images of cross-sections of biological tissue. The OCT technology is mainly used in ophthalmology to examine the retina and the like because it is non-invasive, the light used is harmless to the body, and high-resolution images can be obtained in real time at a relatively low cost. Therefore, it is necessary to research and develop a system for extracting a retinal disease from an OCT image and classifying and visualizing the retinal disease.

### SUMMARY OF THE INVENTION

The present invention is directed to providing a system for providing an artificial intelligence (AI)-based retinal disease diagnosis assistance service that preprocesses tomographic images of the retina captured by an optical coherence tomography (OCT) device, which uses a non-destructive examination method, before inputting the tomographic images into an AI algorithm, causes the AI algorithm to detect and classify a preset retinal disease class in a brightness (B)-scan image, visualizes and outputs detection and classification results to a user terminal, provides a base of the detection and classification to help a medical worker with diagnosis, models the AI algorithm after a process in which medical workers diagnose a retinal disease to improve accuracy, and can be trusted by medical workers through visualization and utilized for actual diagnosis. However, technical objects of the present embodiment are not limited to that described above, and there may be other technical objects.

According to an aspect of the present invention, there is provided a system for providing a diagnosis assistance service, the system including a user terminal configured to transmit tomographic images of a retina captured by an OCT device, which uses a non-destructive examination method, and receive and output a retinal disease classification result and a diagnosis assistance service provision server that includes a preprocessing unit configured to receive and preprocess the tomographic images, a diagnosis assistance unit configured to input the preprocessed tomographic images into a prebuilt AI algorithm, a visualization unit configured to visualize a retinal disease classified by the prebuilt AI algorithm, and a sharing unit configured to provide the retinal disease classified by the prebuilt AI algorithm to the user terminal and transmit a visualization result.

### BRIEF DESCRIPTION OF THE DRAWINGS

The above matters and other objects, features, and advantages of the present invention will become more apparent to those of ordinary skill in the art by describing exemplary embodiments thereof in detail with reference to the accompanying drawings, in which:

FIG. 1 is a diagram illustrating a system for providing an artificial intelligence (AI)-based retinal disease diagnosis assistance service according to an exemplary embodiment of the present invention;

FIG. 2 is a block diagram illustrating a diagnosis assistance service provision server included in the system of FIG. 1;

FIGS. 3A-3J and FIGS. 4A-4O are diagrams illustrating an AI-based retinal disease diagnosis assistance service according to an exemplary embodiment of the present invention; and

FIG. 5 is an operational flowchart illustrating a method of providing an AI-based retinal disease diagnosis assistance service according to an exemplary embodiment of the present invention.

### DETAILED DESCRIPTION OF EXEMPLARY EMBODIMENTS

Hereinafter, exemplary embodiments of the present invention will be described in detail with reference to the accompanying drawings such that those of ordinary skill in the art can readily implement the present invention. However, the present invention may be implemented in various different forms and is not limited to exemplary embodiments set forth herein. In the drawings, elements not relevant to the description are omitted to clearly describe the present invention, and like reference numerals are used for like elements throughout the specification.

Throughout the specification, when any one element is referred to as being “connected to” another element, the elements may be “directly connected” to each other or may be “electrically connected” to each other with still another element interposed therebetween. Also, when a certain element “includes” a certain component, other components are not excluded and may be further included unless otherwise described, and it should be understood that it does not preclude the possibility of addition or presence of one or more other features, numbers, steps, operations, components, parts, or combinations thereof.

The terms “about,” substantially,” and the like used throughout the specification mean figures corresponding to manufacturing and material tolerances specific to the stated meanings and figures close thereto, and are used to prevent unconscionable abusers from unfairly using the disclosure of figures precisely or absolutely described to aid the understanding of the present invention. The term “operation” or “operation of” used throughout the specification of the present invention does not mean “operation for.”

In the present specification, the term “unit” includes a unit implemented by hardware, a unit implemented by software, and a unit implemented by both hardware and software. Further, one unit may be implemented by two or more pieces of hardware, and two or more units may be implemented by one piece of hardware. However, a “unit” is not limited to software or hardware and may be configured to be in an addressable storage medium or configured to reproduce one or more processors. Therefore, as an example, a “unit” includes components such as software components, object-oriented software components, class components, and task components, processes, functions, attributes, procedures, subroutines, segments of program code, drivers, firmware, microcode, circuits, data, databases, data structures, tables, arrays, and variables. Components and functions provided in “units” may be combined into a smaller number of components and “units” or may be separated into additional components and “units.” Moreover, components and “units” may be implemented to reproduce one or more central processing units (CPUs) in a device or a secure multimedia card.

In the present specification, some operations or functions described as performed by a terminal, an apparatus, or a device may be performed instead in a server connected to the terminal, apparatus, or device. Similarly, some operations or functions described as being performed by a server may be performed in a terminal, an apparatus, or a device connected to the server.

In the present specification, some operations or functions described as mapping with or matching a terminal map with or match a unique number of the terminal or personal identification information which is identification data of the terminal.

Hereinafter, the present invention will be described in detail with reference to the accompanying drawings.

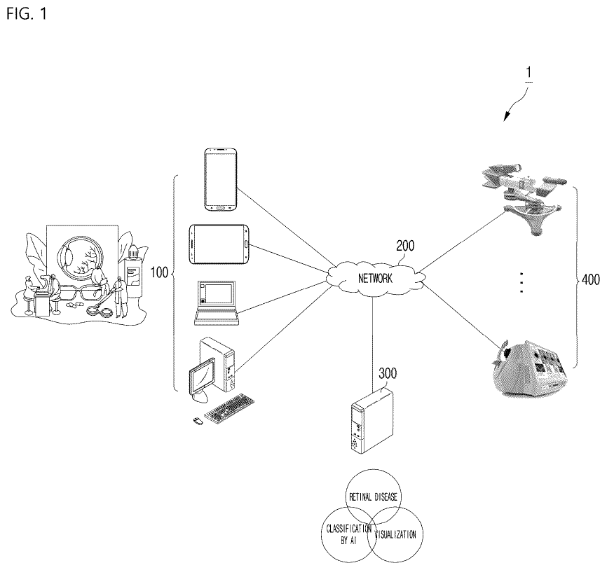

FIG. 1 is a diagram illustrating a system for providing an artificial intelligence (AI)-based retinal disease diagnosis assistance service according to an exemplary embodiment of the present invention. Referring to FIG. 1, a system 1 for providing an AI-based retinal disease diagnosis assistance service may include at least one user terminal 100, a diagnosis assistance service provision server 300, and at least one optical coherence tomography (OCT) device 400. However, the system 1 for providing an AI-based retinal disease diagnosis assistance service shown in FIG. 1 is merely one embodiment of the present invention, and thus the present invention is not interpreted as being limited to FIG. 1.

The components of FIG. 1 are generally connected via a network 200. For example, as shown in FIG. 1, the at least one user terminal 100 may be connected to the diagnosis assistance service provision server 300 via the network 200. Also, the diagnosis assistance service provision server 300 may be connected to the at least one user terminal 100 and the at least one OCT device 400 via the network 200. Further, the at least one OCT device 400 may be connected to the diagnosis assistance service provision server 300 via the network 200.

Here, a network is a connective structure that allows information to be exchanged between individual nodes such as a plurality of terminals and servers. Examples of a network include a local area network (LAN), a wide area network (WAN), the Internet (the world wide web (WWW)), a wired or wireless data communication network, a telephone network, a wired or wireless television communication network, etc. Examples of the wireless data communication network include, but are not limited to, a 3rd Generation (3G) network, a 4th Generation (4G) network, a 5th Generation (5G) network, a 3rd Generation Partnership Project (3GPP) network, a 5th Generation Partnership Project (5GPP) network, a 5G New Radio (NR) network, a 6th Generation of cellular networks (6G) network, a Long Term Evolution (LTE) network, a World Interoperability for Microwave Access (WiMAX) network, a Wi-Fi network, the Internet, a LAN, a wireless LAN, a WAN, a personal area network (PAN), a radio frequency (RF) network, a Bluetooth network, a near-field communication (NFC) network, a satellite broadcast network, an analog broadcast network, a digital multimedia broadcasting (DMB) network, etc.

In the following description, the term “at least one” is defined as a term including the singular and plural meanings. Even when the term “at least one” is not present, each component may be present in a singular or plural form, and it is obvious that the term may have a singular or plural meaning. In addition, the fact that each component is provided in a singular or plural form means that it is changeable in accordance with embodiments.

The at least one user terminal 100 may be a terminal of a medical worker who diagnoses a retinal disease using a webpage, an app page, a program, or an application related to the AI-based retinal disease diagnosis assistance service.

Here, the at least one user terminal 100 may be implemented as a computer that may access a server or a terminal at a remote location via a network. The computer may include, for example, a navigation device, a notebook, a desktop, a laptop, etc., equipped with a web browser. In this case, the at least one user terminal 100 may be implemented as a terminal that may access a server or a terminal at a remote location via a network. The at least one user terminal 100 is, for example, a mobile communication device in which portability and mobility are ensured and may be any type of handheld wireless communication device, such as a Global Positioning System (GPS) terminal, a Personal Communication System (PCS) terminal, a Global System for Mobile Communications (GSM) terminal, a Personal Digital Cellular (PDC) terminal, a Personal Handyphone System (PHS) terminal, a Personal Digital Assistant (PDA) terminal, an International Mobile Telecommunication (IMT)-2000 terminal, a Code Division Multiple Access (CDMA)-2000 terminal, a wideband CDMA (W-CDMA) terminal, a Wireless Broadband Internet (WiBro) terminal, a smartphone, a smartpad, a tablet personal computer (PC), etc.

The diagnosis assistance service provision server 300 may be a server that provides the webpage, the app page, the program, or the application related to the AI-based retinal disease diagnosis assistance service. The diagnosis assistance service provision server 300 may be a server that, when imaging is completed by the OCT device 400, preprocesses the images, inputs the preprocessed images into an AI algorithm as a query to classify the images into a preset retinal disease class and visualizes the location of a lesion in a brightness (B)-scan image to provide the visualized location to the user terminal 100.

The diagnosis assistance service provision server 300 may be implemented as a computer that may access a server or a terminal at a remote location via a network. For example, the computer may be a navigation device, a notebook, a desktop, a laptop, etc., equipped with a web browser.

The at least one OCT device 400 may be a device that transmits tomographic images to the diagnosis assistance service provision server 300 through the user terminal 100 using or without using the webpage, the app page, the program, or the application related to the AI-based retinal disease diagnosis assistance service.

Here, the at least one OCT device 400 may be implemented as a computer that may access a server or a terminal at a remote location via a network. For example, the computer may be a navigation device, a notebook, a desktop, a laptop, etc. equipped with a web browser. Alternatively, the at least one OCT device 400 may be implemented as a terminal that may access a server or a terminal at a remote location via a network. For example, the at least one OCT device 400 is a mobile communication device in which portability and mobility are ensured and may be any type of handheld wireless communication device, such as a GPS terminal, a PCS terminal, a GSM terminal, a PDC terminal, a PHS terminal, a PDA terminal, an IMT-2000 terminal, a CDMA-2000 terminal, a W-CDMA terminal, a WiBro terminal, a smartphone, a smartpad, a tablet PC, etc.

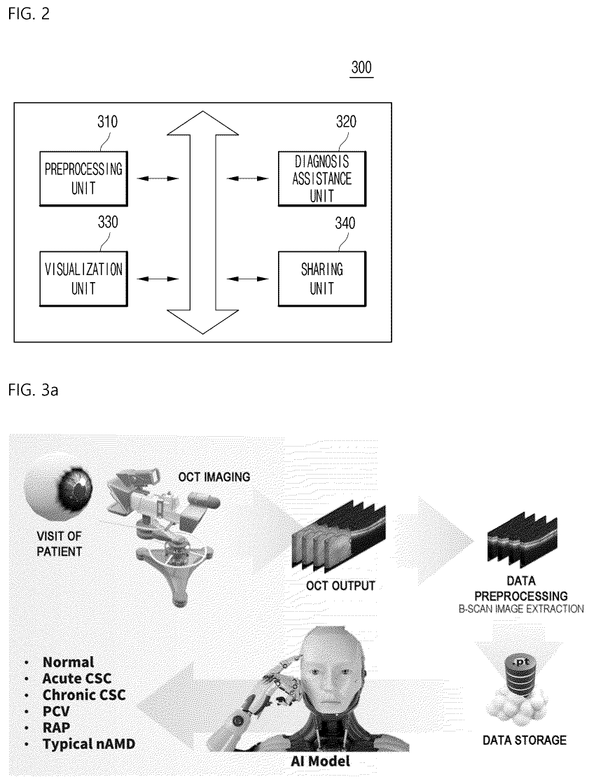

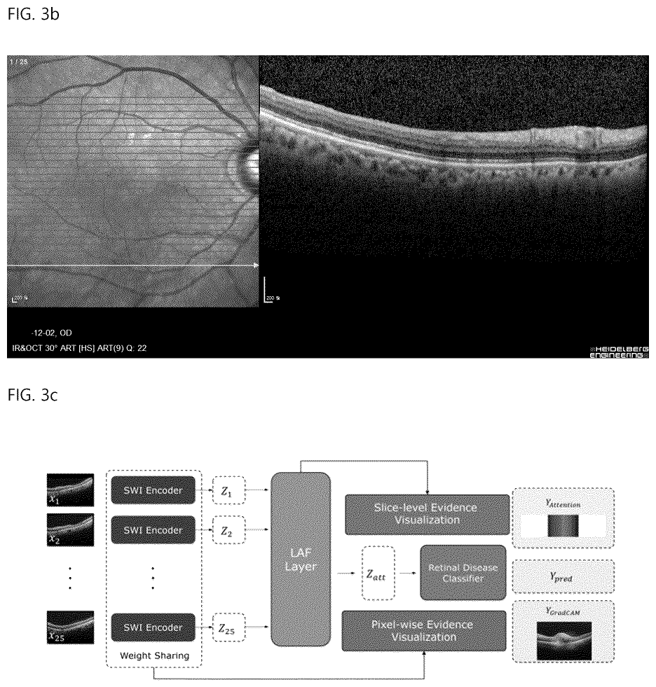

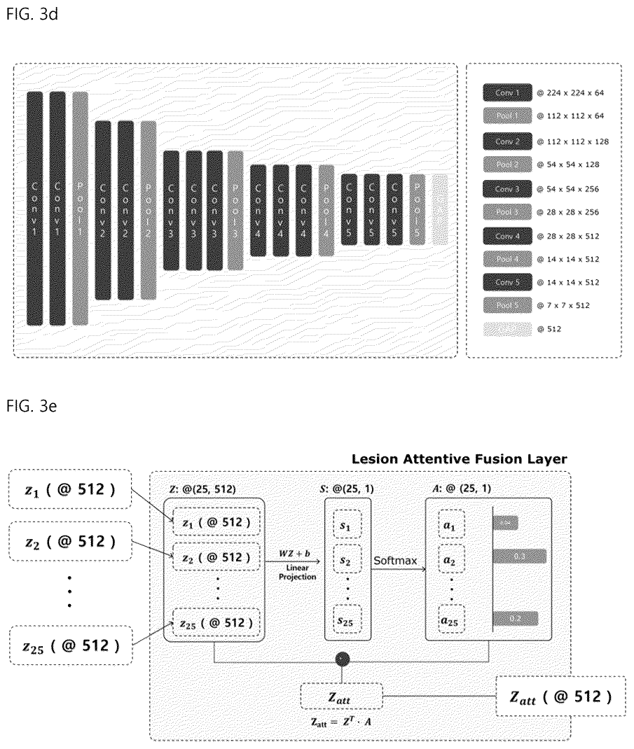

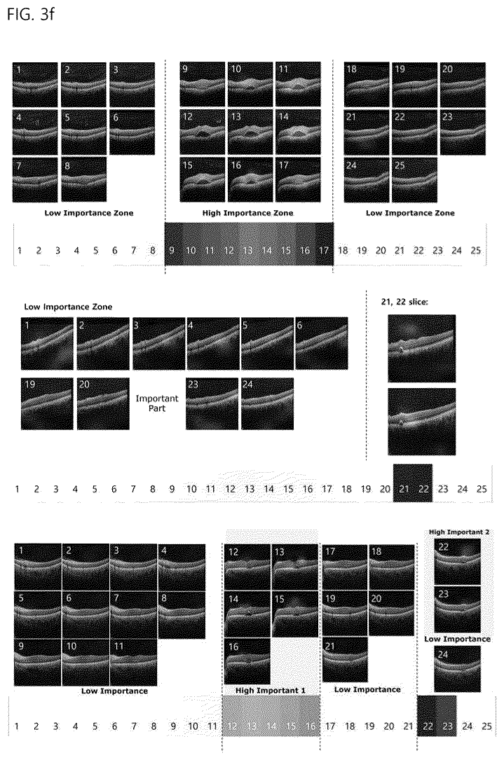

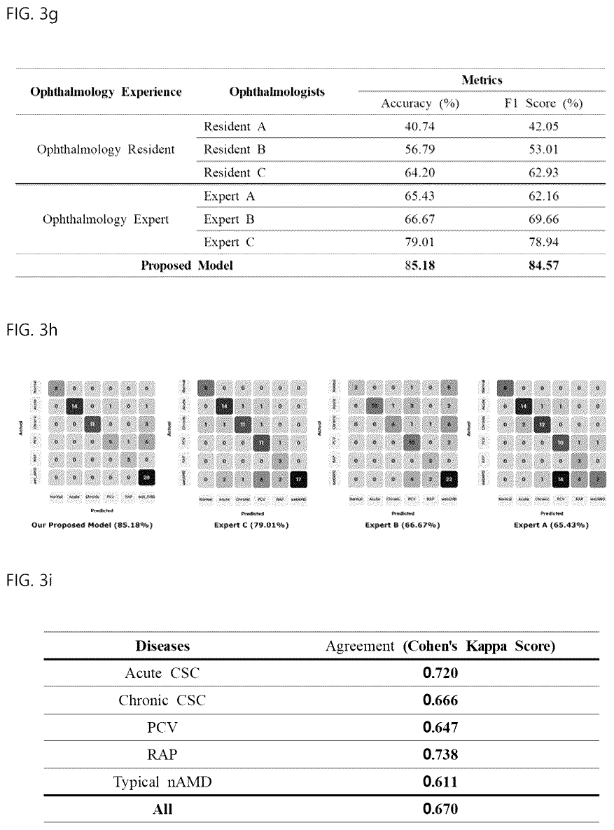

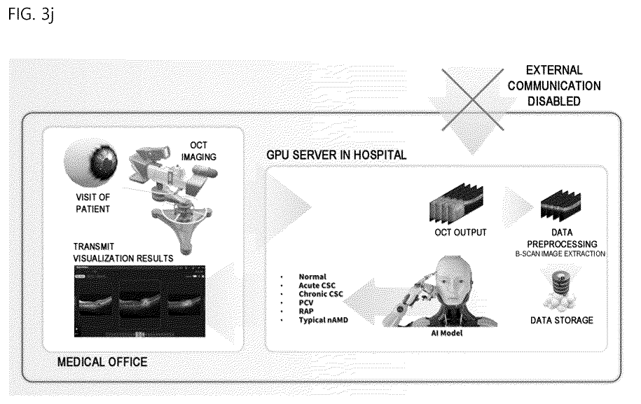

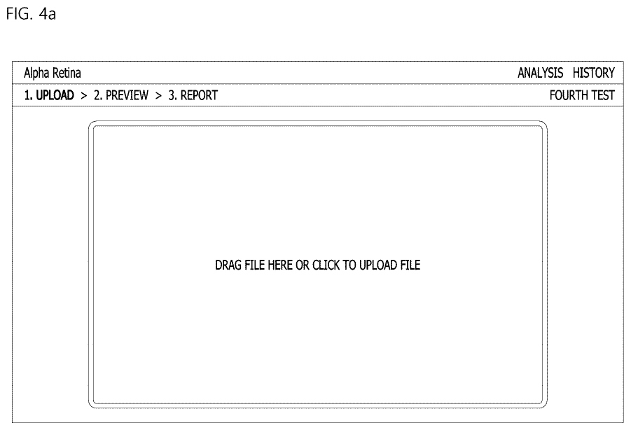

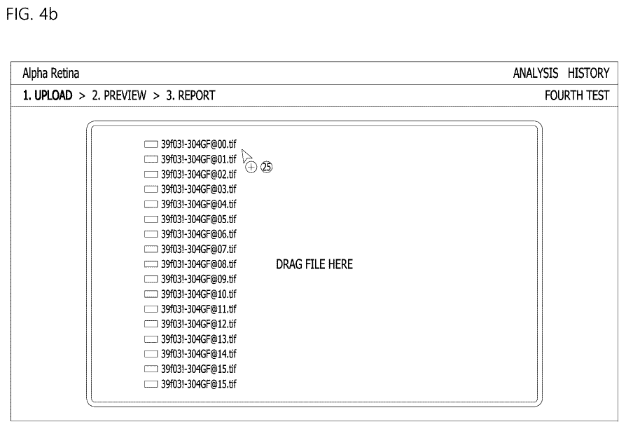

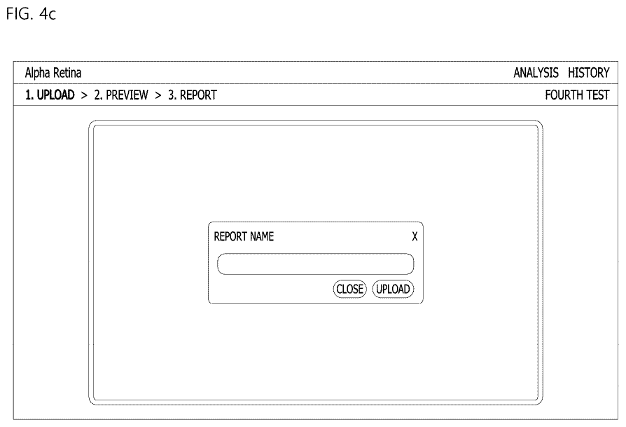

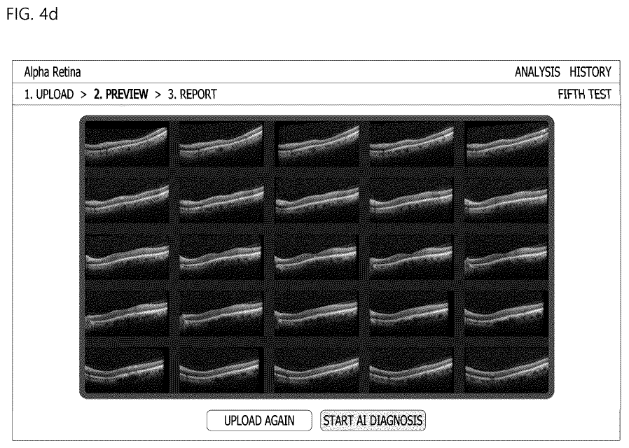

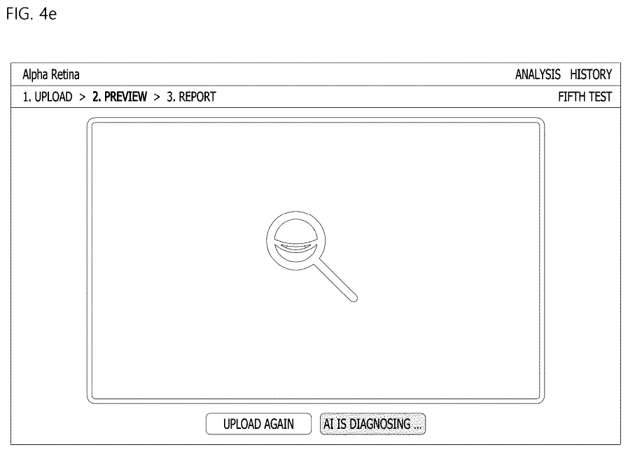

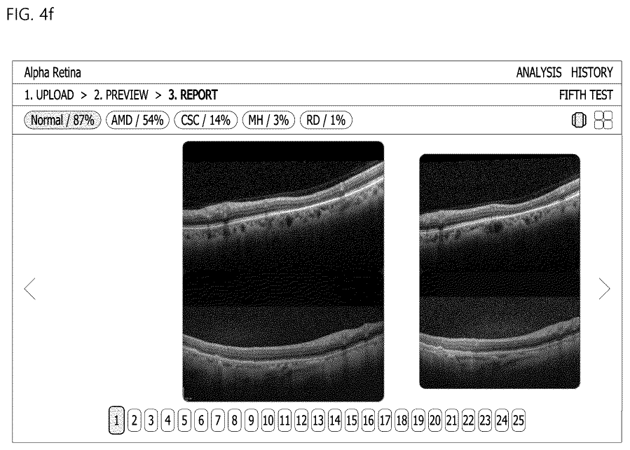

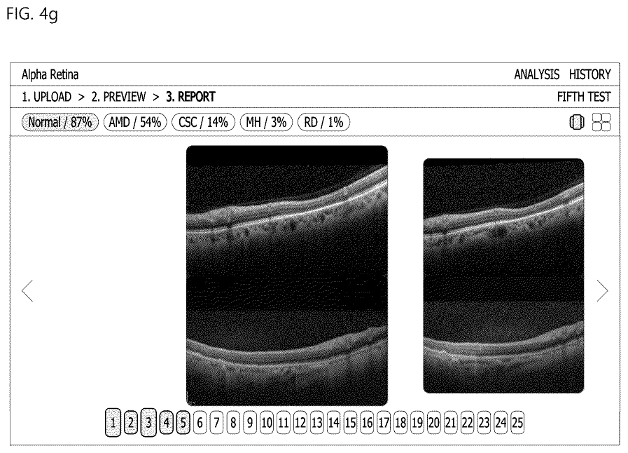

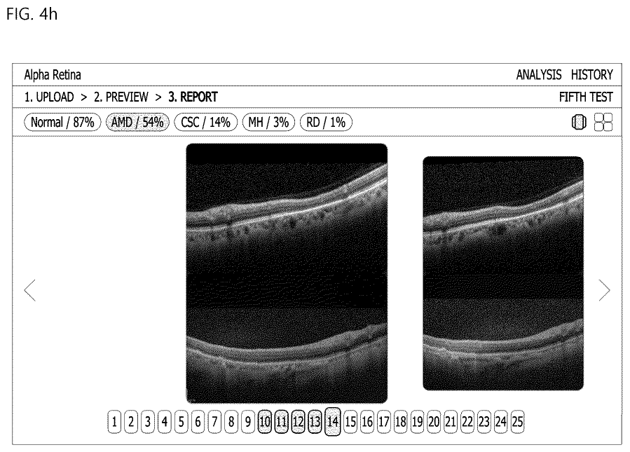

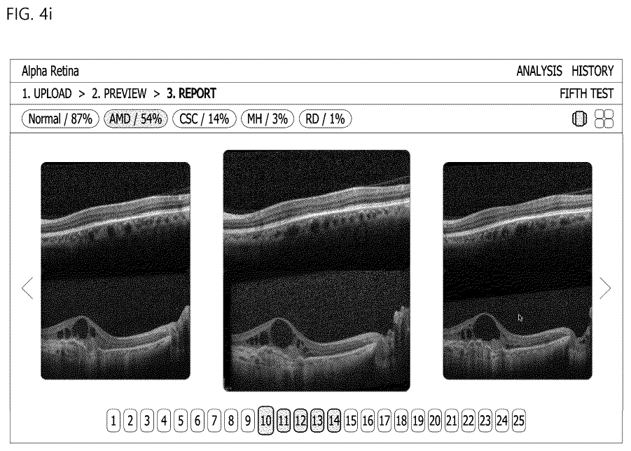

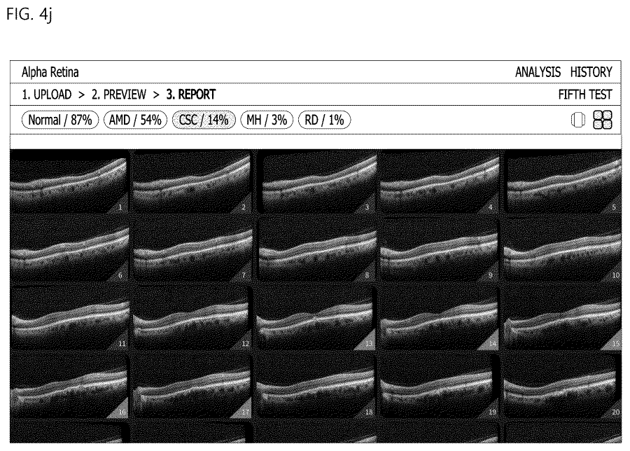

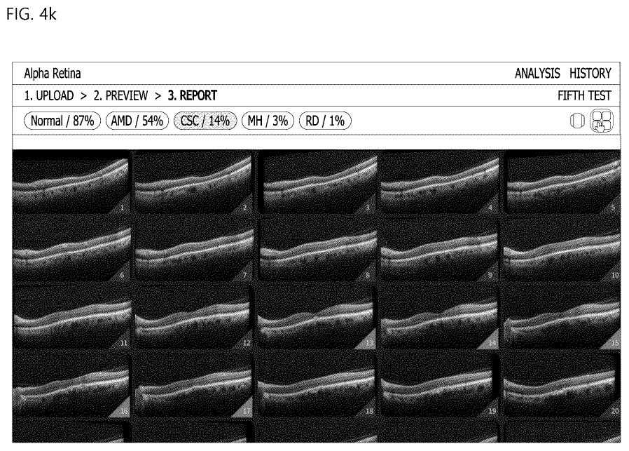

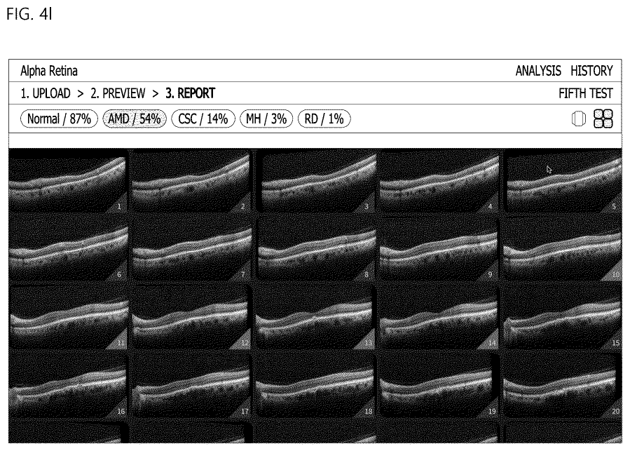

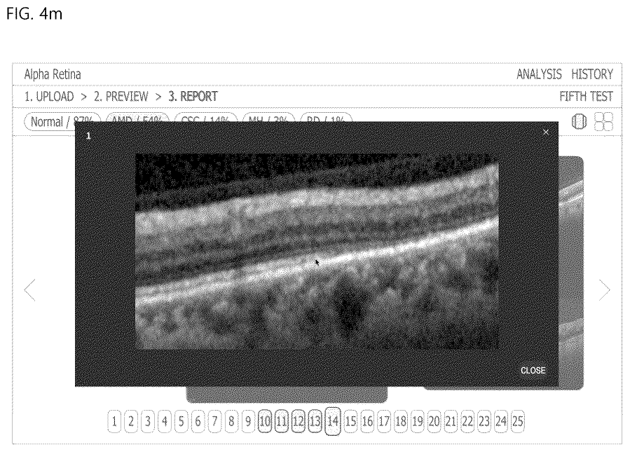

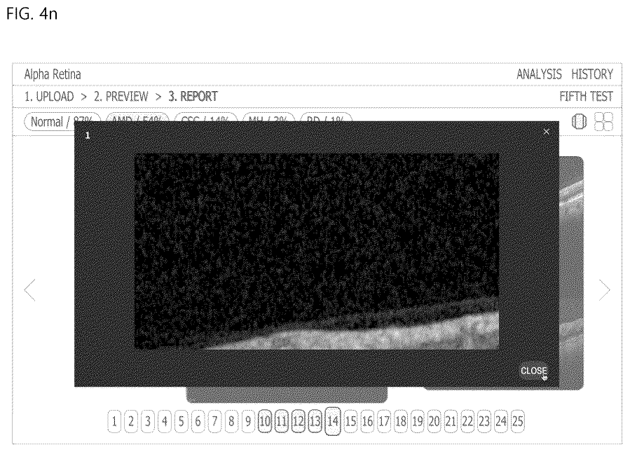

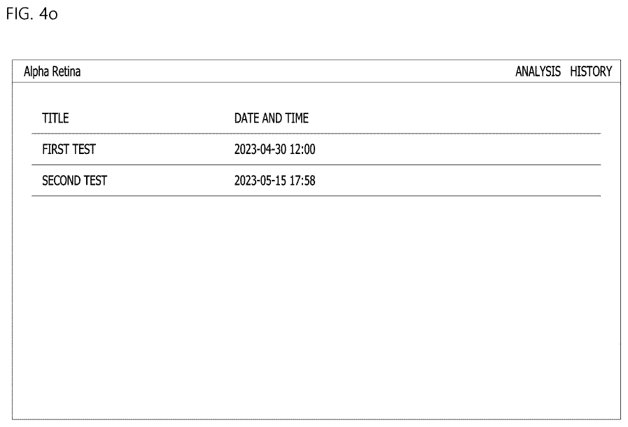

FIG. 2 is a block diagram illustrating a diagnosis assistance service provision server included in the system of FIG. 1, and FIGS. 3 and 4 are diagrams illustrating an AI-based retinal disease diagnosis assistance service according to an exemplary embodiment of the present invention.

Referring to FIG. 2, the diagnosis assistance service provision server 300 may include a preprocessing unit 310, a diagnosis assistance unit 320, a visualization unit 330, and a sharing unit 340.

When the diagnosis assistance service provision server 300 according to the exemplary embodiment of the present invention or another server (not shown) interoperating with the diagnosis assistance service provision server 300 transmits the application, the program, the app page, the webpage, etc., related to the AI-based retinal disease diagnosis assistance service to the at least one user terminal 100 and the at least one OCT device 400, the at least one user terminal 100 and the at least one OCT device 400 may install or open the application, the program, the app page, the webpage, etc., related to the AI-based retinal disease diagnosis assistance service. In addition, a script executed by a web browser may be used to run the service program on the at least one user terminal 100, and the at least one OCT device 400. Here, the web browser is a program that enables a user to use a web (WWW) service, that is, a program that receives and shows hypertext described in hypertext markup language (HTML). For example, the web browser is Chrome, Microsoft Edge, Safari, FireFox, Whale, UC browser, etc. In addition, the application is an application program on a terminal and may be, for example, an app executed on a mobile terminal (smartphone).

A diagnosis assistance process according to an exemplary embodiment of the present invention involves an “imaging-preprocessing-AI algorithm-classification and visualization” process as shown in FIG. 3A. Here, as shown on the right side of FIG. 3B, a B-scan image is utilized among tomographic images. This is an OCT output, that is, a first tomographic image, of one patient. The number of images captured is the number of sections set by an ophthalmologist, but an AI model according to an exemplary embodiment of the present invention is designed with 25 images which are generally used. The OCT output of FIG. 3B also corresponds to a first of 25 images.

The AI algorithm according to an exemplary embodiment of the present invention may be trained to only extract B-scan images from 25 input images when tomographic images are automatically given for training by the OCT device 400. This is intended to imitate a diagnosis process of an ophthalmologist. Although an ophthalmologist views all the 25 images, only a few key images are clinically used for classification. Accordingly, the AI algorithm according to an exemplary embodiment of the present invention also extracts only B-scan images from the 25 images for classification. To train the AI algorithm according to an exemplary embodiment of the present invention, when OCT images are given, only B-scan images are extracted, transformed into tensors of PyTorch, and then stored as “.pt” files. The “.pt” files may be utilized to improve both training speed and efficiency.

In other words, not all the 25 tomographic images are utilized for clinical diagnosis, but only some images in which a lesion is found are utilized to make a diagnosis. Generally, as few as one or two images and as many as ten images are utilized. In particular, central images are mainly utilized. Only B-scan images are extracted and used such that the AI algorithm may imitate such a diagnosis method. In addition, training data may be classified into six classes in total, and an inference model may classify images into the six classes used in training.

TABLE 1

| Class | Retinal disease |
|---|---|
| 1 | Normal |
| 2 | Acute central serous chorioretinopathy (CSC) |
| 3 | Chronic CSC |
| 4 | Polypoidal choroidal vasculopathy (PCV) |
| 5 | Retinal angiomatous proliferation (RAP) |
| 6 | Typical neovascular age-related macular degeneration (nAMD) |

### <Data Preprocessing Pipeline>

The preprocessing unit 310 may receive and preprocess tomographic images. The user terminal 100 may transmit retinal tomography images captured by the OCT device 400 which uses a non-destructive examination method. When the tomographic images are received, the preprocessing unit 310 may extract only B-scan images from the tomographic images, perform data normalization, and then perform tensor transformation to input the extracted B-scan images into a graphics processing unit (GPU). Here, the GPU is the diagnosis assistance service provision server 300 according to an exemplary embodiment of the present invention. In other words, tensor transformation may be performed to input images into the AI algorithm. In brief, as described above, when tomographic images are given by an OCT device, only B-scan images are extracted from among the 25 images, transformed into tensors of PyTorch, and then stored as “.pt” files. During this process, data normalization may be performed, and tensor conversion may be performed for input into a GPU.

### <AI Algorithm>

### <Diagnosis>

Referring to FIG. 3C, the diagnosis assistance unit 320 may input the preprocessed tomographic images into the prebuilt AI algorithm. The prebuilt AI algorithm may include: (1) a slice-wise image (SWI) encoder that generates embeddings, which is summary information of B-scan images, when the B-scan images are input; (2) a lesion-attentive fusion (LAF) layer that automatically detects preset lesion images among the B-scan images, converts the detected lesion images into a one-dimensional (1D) array, and then transmits the 1D array; and (3) a retinal disease classifier that classifies the 1D array input from the foregoing LAF layer into a preset retinal disease class using a fully connected layer.

### <First Diagnosis_SWI Encoder>

Referring to FIG. 3D, the SWI encoder may generate embeddings using a convolutional neural network (CNN)-based image classification model. In other words, when 25 tomographic images are input, the SWI encoder generates embeddings which are summary information of each of the images, and the CNN-based image classification model may be utilized to generate the embeddings. Summary information of tomographic images is generated by one encoder through parameter sharing. In this way, 25 embeddings are generated in total.

Here, a CNN is a useful algorithm for detecting a pattern for analyzing an image, learns images directly, and classifies an image using a pattern. According to an exemplary embodiment of the present invention, a CNN model such as a visual geometry group (VGG)-16 neural network, a VGG-19 neural network, a residual neural network (ResNet), or a densely connected convolutional neural network (DenseNet) may be used, and one with the highest accuracy among the CNN models may be set as a model according to an exemplary embodiment of the present invention. The VGG neural networks are classified by the number of layers. All convolutional layers of a VGG neural network use a 3×3 filter with a stride of 1. When several 3×3 filters are used, it is possible to use two or three rectified linear units (ReLUs) instead of one and reduce the number of parameters. With an increase in the number of layers, non-linearity increases, which may increase the discriminability of features of the model.

The ResNet includes convolution blocks and residual blocks. Since the residual blocks use batch normalization, the problem of covariate shift is relieved. When there is an input image, a 7×7 convolutional operation is performed, and a 3×3 max pooling operation is performed. Subsequently, a feature map generated through intermediate layers of four stages is handed over to the last layer. The handed feature map is used to perform an average pooling operation, and then the result is handed to a fully connected layer. The DenseNet includes dense blocks and transition blocks. In the dense blocks, all preceding layers are connected. Accordingly, there is little information loss, preceding features can be reused, and the problem of gradient vanishing is improved. However, various CNN models may be used in addition to the described CNN models, and then a model with the highest accuracy may be selected.

### <Second Diagnosis_LAF Layer>

Referring to FIG. 3E, through an attention structure, the LAF layer automatically detects lesion images corresponding to “important” images used for actual diagnosis among the 25 tomographic images, calculates a weighted sum of the lesion images in accordance with importance, contracts the tomographic images once again, and transmits the result to a next layer. In this way, a two-dimensional (2D) array corresponding to (25 embeddings×embedding size) is changed into a 1D array with a size of (embedding size×1).

### <Third Diagnosis_Retinal Disease Classifier>

A fully connected layer is used to classify the 1D array input from the LAF layer into one of six classes in total. As shown in Table 1 above, the preset retinal disease classes may include: (1) a normal class; (2) an acute CSC class; (3) a chronic CSC class; (4) a PCV class; (5) a RAP class; and (6) a typical nAMD class. In other words, according to an exemplary embodiment of the present invention, an AI model can be built to distinguish chronic CSC and acute CSC which are subtypes of CSC and AMD corresponding to the world's four major retinal diseases, PCV and RAP which are subtypes of nAMD, typical nAMD excluding PCV and RAP, and normal eyes from each other, and a pipeline can be built to facilitate use of the AI model.

### <Visualization>

The visualization unit 330 may visualize a retinal disease classified by the prebuilt algorithm. The visualization unit 330 may include a pixel-wise evidence visualization (PEV) module for detecting and visualizing the location of a lesion in B-scan images and a slice-wise evidence visualization (SEV) module for performing slice-wise visualization on the B-scan images. Here, the PEV module may visualize the location of the lesion using a gradient-weighted class activation map (Grad-CAM) technique. The heat maps on the upper side of FIG. 3F are PEV visualization results, and the color bands on the lower side are SEV visualization results.

### <First Visualization_PEV>

The PEV module performs pixel-wise visualization as shown on the upper side of FIG. 3F. According to an exemplary embodiment of the present invention, the Grad-CAM technique is used to detect and visualize the location of a lesion in one B-scan image. According to the Grad-CAM technique, gradients of a classification score with respect to the final convolutional feature map are used to determine a part of an input image that has the greatest influence on the classification score. A part with the largest gradient is a part of the corresponding data that affects the final score the most. As shown on the heat maps on the upper side of FIG. 3F, a framework according to an exemplary embodiment of the present invention accurately detects locations in which a lesion is found in B-scan images because there is a hole in the images. When the color of a heat map is closer to red, there is a higher possibility of a lesion.

### <Second Visualization_SEV>

The SEV module performs slice-wise visualization as shown on the lower side of FIG. 3F, which shows colored bands. When the color of a band is closer to blue, there is a higher possibility of a lesion being in the corresponding image. It is possible to see that the AI algorithm accurately detects a part to be significantly considered in the diagnosis process.

The significance of this visualization starts from the fact that existing AI algorithms simply informs of a disease and no more and thus medical workers do not know how and on what basis the AI algorithm diagnoses or classifies the disease, resulting in a lack of trust in the AI result, making the existing AI algorithms difficult to use in actual clinical practice. Further, in clinical practice, classification is often performed on a few key images rather than all images, but the existing AI algorithms consider all images equally without considering that. Therefore, the AI algorithm according to an exemplary embodiment of the present invention improves accuracy by imitating a process in which a doctor makes a diagnosis in clinical practice, can gain the trust of medical workers by presenting the evidence through visualization, and can be useful in actual diagnosis.

### <Performance of AI Algorithm>

To verify the performance of the AI algorithm according to an exemplary embodiment of the present invention, diagnosis results of the AI algorithm were compared with those of medical workers. Referring to FIGS. 3G and 3H, ophthalmology residents were composed of ophthalmologists with four years of clinical experience or less, and ophthalmology experts were composed of retinal specialists with 10 year of experience or more. The AI algorithm according to an exemplary embodiment of the present invention outperformed the ophthalmology experts and particularly showed the maximum accuracy difference of two times or more from the less experienced ophthalmologists. FIG. 3H shows confusion matrices for comparison with ophthalmology experts. Referring to FIG. 3I, Expert C who was a retinal specialist making the most accurate classifications was additionally asked to select slides that were significantly considered during the experimental process, and the selected slides were comparatively analyzed with SEV results of the AI algorithm according to an exemplary embodiment of the present invention using Cohen's Kappa scores. As a result, a Cohen's Kappa score of 0.67 was derived, which represents substantial agreement. This means that the AI algorithm significantly considers images that are actually used for classification by medical workers to perform analysis and diagnose a corresponding person with a disease.

### <Clinical Software>

The sharing unit 340 may provide a retinal disease classified by the prebuilt AI algorithm to the user terminal 100 and transmit visualization results. The user terminal 100 may receive and output the retinal disease classification result. Software shown in FIGS. 4A to 4O was developed such that the AI algorithm and framework according to an exemplary embodiment of the present invention may be utilized in clinical practice. As shown in FIG. 3J, the system according to an exemplary embodiment of the present invention is divided into the user terminal 100 in a medical office and the diagnosis assistance service provision server 300 which is a GPU server in a hospital. When a patient's OCT images are taken in the medical office, the diagnosis assistance service provision server 300 processes and analyzes the data to make a diagnosis and transmits the results to the software of the user terminal 100 which is a computer in the medical office. The software is web-based software and allows a user to see visualization results in various ways. In addition, the corresponding structure is manufactured not to require communication with the outside of the system in order to protect personal information of patients. All images and AI inferences are processed and stored by the diagnosis assistance service provision server 300 which is the hospital server.

Although the diagnosis assistance service provision server 300 is not set for communication with the outside in FIG. 3J, there may be a case where it is necessary to cooperate with an external medical worker, a case where a medical worker may remotely work from home, a case where it is necessary to transmit electronic medical records (EMRs) to the outside, and the like. Accordingly, virtual desktop infrastructure (VDI) (virtualization) may be achieved using desktop as a service (DaaS).

### <Cloud>

Cloud and virtualization technologies may be combined to handle work in the field and upload the results using a mobile device. When VDI is used, a medical worker may use an external terminal such as a notebook like the user terminal 100, but actual work may be done by the sharing unit 340. This enables a user to handle work through a virtual terminal using a mobile device in the field and enables efficient management. Also, sample management is processed in real time to increase the level of statistics management, and it is possible to provide services such as cloud-based product prediction, simulation, and the like. VDI technology, the pinnacle of the cloud, has reached the point where a virtual desktop environment is customized for each medical worker via the cloud. An individual virtualization desktop provided by the sharing unit 340 may implement any program including an operating system (OS) and an application-based work environment while handling processes and data within the cloud platform. A medical worker is simply required to remotely access the sharing unit 340, send inputs through the user terminal 100, and display processing results on the display of the user terminal 100. Since main tasks and computational processing are performed on the server, the user terminal 100 does not require high performance, and all data need not be stored on the user terminal 100 because each medical worker's individual work and storage space is implemented through the cloud platform in a data center. Here, the user terminal 100 merely serves as a terminal for remote access, ensuring the reliability of patching, maintaining, and recovering the user terminal 100, and remarkably reducing the time and effort for managing existing PCs in an integrated manner. Centralized control is enhanced to enable unified authentication and access management, all logs can be tracked in real time, and no data is stored on the user terminal 100. Accordingly, VDI is highly suitable for environments requiring high security.

According to an exemplary embodiment of the present invention, when research and development are managed, a method of providing VDI through DaaS using a desktop experience (DeX) function may be further utilized. On the basis of a mobile network connected using 3G secure communication (International Telecommunications Union-Telecommunication (ITU-T) international standard) technology, a mobile terminal may access a secure network access point (AP) using a secure universal subscriber identity module (USIM), and authentication is made using a certificate and biometric information of the user terminal 100 on the basis of a white list. When medical worker authentication of the user terminal 100 fails or the user terminal 100 is identified as a stolen user device, the corresponding USIM and the user terminal 100 may be remotely locked and deleted. After the user terminal 100 passes medical worker authentication, the application is run in a secure domain, and data is encrypted using a private key of the user to communicate with a cloud VDI server. To utilize DaaS of the cloud, a medical worker may build a PC environment by utilizing a smartphone DeX function without any spatial limitation. Here, DeX is a technology that Samsung introduced in 2017 to connect a smartphone to a monitor and provide a work environment similar to the Windows OS of a regular PC. Since the PC environment built using DeX simply serves as a terminal for VDI access, sample development can be conducted and managed on the basis of the performance of the cloud rather than high performance. In addition, since no data is stored in the user terminal 100, the risk of personal information leakage and hospital confidentiality leakage is eliminated.

### <Prediction of Retinal Disease>

A multi-layer perceptron (MLP) may be utilized to predict whether a patient will develop a retinal disease on the basis of his or her disease code. Alternatively, disease codes may be listed in historical logs to create time-series data, which may be used to predict whether a patient will develop a retinal disease. In other words, disease codes related to retinal diseases are extracted by extracting correlations on the basis of machine learning, and when a disease code related to a retinal disease is extracted from a patient's disease codes, time-series data is generated using only the related disease code and then analyzed to predict whether the patient will develop the retinal disease. For example, it is assumed that there are A-B-C-D-E disease codes, some of the disease codes associated with retinal diseases are A-C-D-E, and when a person goes through the process of A-C-D-E, he or she develops a retinal disease, specifically, nAMD. In this case, a patient who is currently in the A-C-D-E process may be warned of the possibility of progression to macular degeneration, counseled to quit smoking, aggressively advised to manage his or her blood pressure, hyperlipidemia, and cardiovascular disease, and encouraged to maintain a healthy weight, wear sunglasses when outdoors, eat plenty of green vegetables and blue fish, take eye nutrients for dry macular degeneration, and have regular eye exams and treatment, thereby delaying the development as much as possible.

An operational process according to the configuration of the diagnosis assistance service provision server of FIG. 2 described above will be described in detail below with reference to FIGS. 3A-3J and FIGS. 4A-4O. However, this embodiment is merely one of various embodiments of the present invention, and the present invention is not limited thereto.

FIGS. 3A-3J show an overview, preprocessing, the AI algorithm, visualization, and performance evaluation as described in FIG. 2, and the description will not be repeated. The software mentioned in FIG. 3J is disclosed in FIGS. 4A to 4O. Software according to an exemplary embodiment of the present invention (tentatively titled “Alpha Retina”) as shown in FIG. 4A has a menu of 1. Upload, 2. Preview, and 3. Report. Files, that is, 25 tomographic images, are dragged and dropped or clicked in 1. Upload as shown in FIG. 4B, and a report is named as shown in FIG. 4C.

Subsequently, while 25 tomographic images are displayed on one screen as shown in FIG. 4D, a user confirms that the 25 tomographic images are input without any problem and clicks “Start AI diagnosis” in Preview. Then, diagnosis begins as shown in FIG. 4E, and interpretation results of normal, AMD, CSC, macular hole (MH), retinal detachment (RD), and the like are displayed as shown in FIG. 4F. Here, when “Normal” is clicked, normal images are displayed on the upper side (PEV) and the lower side (SEV) as shown in FIG. 4G. Results of AMD are visualized in FIGS. 4H and 4I, results of CSC are visualized in FIG. 4J, and results of AMD and the like are visualized in FIG. 4L. When one of the images is selected, the image is enlarged and pops up as shown in FIG. 4M, and the pop-up image may be further enlarged and moved for examination as shown in FIG. 4N. In addition, an analysis history is provided as a log as shown in FIG. 4O.

Since the above-described method of providing an AI-based retinal disease diagnosis assistance service shown in FIGS. 2, FIGS. 3A-3J and FIGS. 4A-4O is the same as or may be readily inferred from the method of providing an AI-based retinal disease diagnosis assistance service described in FIG. 1, other description will be omitted.

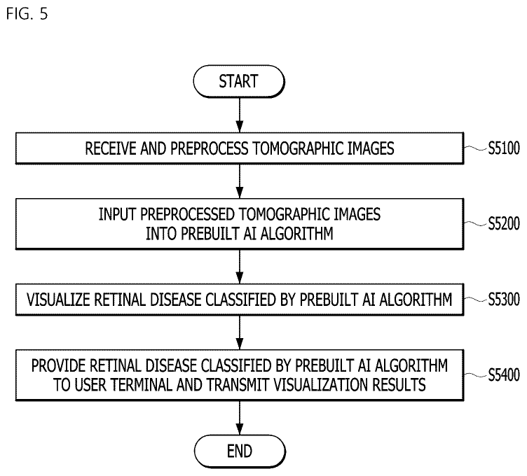

FIG. 5 is a flowchart illustrating a process of transmitting and receiving data between components included in the system for providing an AI-based retinal disease diagnosis assistance service according to an exemplary embodiment of the present invention shown in FIG. 1. An example of the process of transmitting and receiving data between the components will be described below with reference to FIG. 5. However, the present invention is not interpreted as being limited thereto, and it is apparent to those of ordinary skill in the art that the process of transmitting and receiving data shown in FIG. 5 may be modified in accordance with the variety of embodiments described above.

Referring to FIG. 5, a diagnosis assistance service provision server receives and preprocesses tomographic images (S5100).

Then, the diagnosis assistance service provision server inputs the preprocessed tomographic images into a prebuilt AI algorithm (S5200) and visualizes a retinal disease classified by the prebuilt algorithm (S5300).

The diagnosis assistance service provision server provides the retinal disease classified by the prebuilt AI algorithm to a user terminal and transmits visualization results (S5400).

The above-described order of operations S5100 to S5400 is illustrative, and the present invention is not limited thereto. In other words, the above-described order of operations S5100 to S5400 may be changed, and some of the operations may be simultaneously performed or removed.

Since the above-described method of providing an AI-based retinal disease diagnosis assistance service shown in FIG. 5 is the same as or may be readily inferred from the method of providing an AI-based retinal disease diagnosis assistance service described in FIGS. 1 to 4, other description will be omitted.

The method of providing an AI-based retinal disease diagnosis assistance service according to the exemplary embodiment described with reference to FIG. 5 may be implemented in the form of a recording medium including computer-executable instructions such as an application or program module executed by a computer. A computer-readable medium may be any available medium that may be accessed by a computer and includes all of volatile and non-volatile media and removable and non-removable media. Also, the computer-readable medium may include all computer storage media. The computer storage medium includes all of volatile and non-volatile media and removable and non-removable media implemented using any method or technology for storage of information such as computer-readable instructions, data structures, program modules, or other data.

The method of providing an AI-based retinal disease diagnosis assistance service according to the exemplary embodiment of the present invention described above may be executed by an application (which may include a program included in a platform, an OS, or the like basically installed in a terminal) that is basically installed in a terminal, and may be executed by an application (i.e., program) installed directly on a master terminal by a user through an application provision server such as an application store server, an application, or a web server related to the corresponding service. In this sense, the method of providing an AI-based retinal disease diagnosis assistance service according to the exemplary embodiment of the present invention described above may be implemented as an application (i.e., program) basically installed in a terminal or directly installed by a user, and may be recorded on a medium that is readable by a computer such as a terminal or the like.

According to any one of the solutions described above, tomographic images of the retina captured by an OCT device, which uses a non-destructive examination method, are preprocessed before being input into an AI algorithm, the AI algorithm detects and classifies a preset retinal disease class in a B-scan image, and detection and classification results are visualized and output to a user terminal. In this way, the solution can help a medical worker with diagnosis by providing a base of the detection and classification, improve accuracy by modeling the AI algorithm after a process in which medical workers diagnose a retinal disease, and can be trusted by medical workers through visualization and utilized for actual diagnosis.

The above description of the present invention is for illustrative purposes, and those of ordinary skill in the art will understand that the present invention may be easily modified into other specific forms without changing the technical spirit or essential features of the present invention. Therefore, it is to be understood that the embodiments described above are illustrative rather than being restrictive in all aspects. For example, each component described as singular may be implemented in a distributed manner, and similarly, components described as distributed may be implemented in a combined form.

It is to be understood that the scope of the present invention will be defined by the claims rather than the above detailed description and all modifications and alterations derived from the meaning and scope of the claims and their equivalents fall within the scope of the present invention.

1. A system for providing an artificial intelligence (AI)-based retinal disease diagnosis assistance service, the system comprising:
a user terminal configured to transmit tomographic images of a retina captured by an optical coherence tomography (OCT) device, which uses a non-destructive examination method, and receive and output a retinal disease classification result; and
a diagnosis assistance service provision server including a preprocessing unit configured to receive and preprocess the tomographic images, a diagnosis assistance unit configured to input the preprocessed tomographic images into a prebuilt AI algorithm, a visualization unit configured to visualize a retinal disease classified by the prebuilt AI algorithm, and a sharing unit configured to provide the retinal disease classified by the prebuilt AI algorithm to the user terminal and transmit a visualization result, wherein the prebuilt AI algorithm comprises:
a slice-wise image (SWI) encoder configured to generate embeddings, which are summary information of the preprocessed tomographic images, based on the preprocessed tomographic images are input;
a lesion-attentive fusion (LAF) layer configured to automatically detect preset lesion images among the preprocessed tomographic images, convert the detected lesion images into a one-dimensional (1D) array, and then transmit the 1D array; and
a retinal disease classifier configured to classify the 1D array input from the LAF layer into a preset retinal disease class using a fully connected layer,
wherein the SWI encoder generates the embeddings using a convolutional neural network (CNN)-based image classification model comprising a plurality of layers, each layer connected to every preceding layer of the plurality of layers, such that accuracy of the prebuilt AI algorithm is improved by reducing information loss, and improving gradient vanishing.

2. The system of claim 1, wherein, when the tomographic images are received, the preprocessing unit only extracts brightness (B)-scan images from the tomographic images, performs data normalization, and then performs tensor transformation to input the extracted B-scan images into a graphics processing unit (GPU).

3. (canceled)

4. (canceled)

5. The system of claim 1, wherein the preset retinal disease classes comprise:
a normal class;
an acute central serous chorioretinopathy (CSC) class;
a chronic CSC class;
a polypoidal choroidal vasculopathy (PCV) class;
a retinal angiomatous proliferation (RAP) class; and
a typical neovascular age-related macular degeneration (nAMD) class.

6. The system of claim 2, wherein the visualization unit comprises:
a pixel-wise evidence visualization (PEV) module configured to detect and visualize a location of a lesion in the B-scan images; and
a slice-wise evidence visualization (SEV) module for performing slice-wise visualization on the B-scan images.

7. The system of claim 6, wherein the PEV module visualizes the location of the lesion using a gradient-weighted class activation map (Grad-CAM) technique.
